(19) United States
(12) Patent Application Publication
(10) Pub. No.: US 2010/0129906 A1
(43) Pub. Date: May 27, 2010

(54) Method for Obtaining Xeno-Free Hbs Cell line

(75) Inventors: Henrik Semb, Bjarred (SE); Eva Karin Kilmare, Goteborg (SE); Raimund Strehl, Goteborg (SE); Catharina Ellerström, Vastra Frolunda (SE); Katarina Frej, Goteborg (SE); Karina Moya, Molnlycke (SE); Sven Johan Hyllner, Molndal (SE)

Correspondence Address: BUCHANAN, INGERSOLL & ROONEY PC, POST OFFICE BOX 1404, ALEXANDRIA, VA, 22313-1404, US

(73) Assignee: Cellartis AB, Goteborg (SE)

(21) Appl. No.: 12/083,192

(22) PCT Filed: Oct. 6, 2006
(86) PCT No.: PCT/EP2006/009697

§ 371 Date: Feb. 12, 2009

Related U.S. Application Data
(60) Provisional application No. 60/724,815, filed on Oct. 11, 2005.

(30) Foreign Application Priority Data
Oct. 7, 2005 (DK) PA2005 01413

(51) Int. Cl. C12N 5/071

(52) U.S. Cl. 435/366; 435/373

(57) ABSTRACT

A method for obtaining a stable xeno-free hBS cell line, xeno-free hBS cell lines obtained according to said method and use thereof. The method comprises the steps of:
i) removing the zona pellucida from a blastocyst to obtain trophectoderm-enclosed inner cell mass by a xeno-free procedure,
ii) at least partly removing the trophectoderm to obtain isolated inner cell mass cells by a xeno-free procedure,
iii) placing the inner cell mass cells on a layer of human feeder cells in a xeno-free medium,
iv) co-culturing of the inner cell mass cells with human feeder cells for a time period of from about 5 days to about 50 days in a xeno-free medium,
v) releasing the inner cell mass cells or cells derived thereof from trophectoderm overgrowth, if any, by a xeno-free procedure,
vi) selectively, transferring the inner cell mass cells or cells derived thereof to a fresh layer of human feeder cells in a xeno-free medium to obtain xeno-free hBS cells,
vii) propagating the xeno-free hBS cells by co-culturing with human feeder cells in a xeno-free medium to obtain a xeno-free hBS cell line.

The xeno-free hBS cell line is suitable for use in medicine and in in vitro testing.

### FIELD OF THE INVENTION

The present invention relates to a method to obtain a stable xeno-free hBS cell line, xeno-free hBS cell lines obtained according to said method and use thereof.

### BACKGROUND OF THE INVENTION

A stem cell is a cell type that has a unique capacity to renew itself and to give rise to specialized or differentiated cells. Although most cells of the body, such as heart cells or skin cells, are committed to conduct a specific function, a stem cell is uncommitted until it receives signals to develop into a specialized cell type. What makes the stem cells unique is their proliferative capacity, combined with their ability to become specialized. For years, researchers have focused on finding ways to use stem cells to replace cells and tissues that are lost, damaged or diseased. So far, most research has focused on two types of stem cells, embryonic and somatic stem cells. Embryonic stem cells are derived from the pre-implanted fertilized oocyte, i.e. blastocyst, whereas the somatic stem cells are present in the adult organism, e.g. within the bone marrow, epidermis and intestine. Pluripotency tests have shown that whereas the embryonic or blastocyst-derived stem cells (hereafter referred to as blastocyst-derived stem cells or BS cells) can give rise to all cells in the organism, including the germ cells, somatic stem cells have a more limited repertoire in descendent cell types.

Perhaps the most far-reaching potential application of hBS cells is the generation of cells and tissue that could be used for so-called cell therapies. Many diseases and disorders result from disruption of cellular function or destruction of tissues of the body. Today, donated organs and tissues are often used to replace ailing or destroyed tissue. Unfortunately, the number of people suffering from disorders suitable for treatment by these methods by far outstrips the number of organs available for transplantation. The availability of hBS cells and the intense research on developing efficient methods for guiding these cells towards different cell fates, e.g. insulin-producing β-cells, cardiomyocytes, and dopamine-producing neurons, holds growing promise for future applications in cell-based treatment of degenerative diseases, such as diabetes, myocardial infarction and Parkinson's.

However, all currently available human blastocyst-derived stem cell (hBS) lines have at some point during their derivation and/or maintenance been directly or indirectly exposed to animal material. One potential consequence of using xeno-contaminated hBS cells in patients is an increased likelihood of graft rejection [Martin J M et al, 2005]. Moreover, xeno-exposure increases the risk of transferring non-human pathogens in any clinical application. Thus, the predicted hazards associated with using xeno-exposed hBS cells in patients, makes these cells unsuitable for clinical applications. In an attempt to overcome these problems, several groups have used feeder-free matrices [Xu C et al, 2001], or feeder cells of human origin for derivation [Richards M et al, 2002, Inzunza J et al] and culture [Richards M et al, 2003] of hBS cell lines. However, it has so far not been possible to derive and continuously culture hBS cell lines in a completely xeno-free system. In order to obtain and culture hBS cells completely xeno-free, three major hurdles have to be overcome. First, a protocol for the derivation of new hBS cell lines under xeno-free conditions must be developed. Isolation of the inner cell mass (ICM) is classically performed by immunosurgery, a procedure that includes the use of guinea pig serum. We have earlier reported [Heins et al, 2004] that the immunosurgery procedure can be excluded and isolation of the ICM cells can therefore be performed without exposure to animal components. Second and third, a xeno-free feeder culture system in combination with the use of a xeno-free medium is necessary. Since it is not the base medium which causes xeno-contamination, but the commonly used fetal bovine serum (FBS) or serum replacement which contains various animal proteins we decided to test if hBS cells could be cultured for an extended time in human serum. Previous reports on the use of human serum for hBS cell cultures are very few and only indicate short-term culture stablility, i.e. for less than 10 passages [Richards et al, 2002 and 2004]. The difficulties reported by others to maintain hBS cells in an undifferentiated state using human serum supplemented medium initially encouraged the present inventors to instead of developing a hBS cell culture system using human and synthetic components, to seek to develop completely defined culture conditions, i.e. feeder-free cultures and culture medium with completely known compositions and concentrations (defined culture media). However, the present inventors have been able to develop a system for propagation of hBS cells based on human fibroblasts and the serum-comprising culture medium as herein described. hBS cell line SA121 (established according to the procedure of WO2003055992) has now been successfully cultured for over 50 passages in this serum based medium.

Furthermore, the present inventors have developed a system for derivation and culture of completely xeno-free human feeder cells. The existence of xeno-free human feeder cells, such as human foreskin fibroblast feeder cells have been reported before, although the feeders in many cases have been in contact with animal material, such as FBS during the actual establishment and culture of the feeder cells.

In the present invention is reported the successful development of a completely xeno-free system for derivation and maintenance culture of hBS cells using only components meeting the regulatory requirements, such as human or synthetically derived components. The methods herein described completely circumvent any direct or in-direct exposure to animal components whereby completely xeno-free hBS cells can be obtained to be further used as an unlimited source for the development of cell therapies but also for other applications, such as in drug discovery and development, in toxicity testings, for manufacture of medicaments, such as antibodies and vaccines.

### DETAILED DESCRIPTION OF THE INVENTION

The present invention relates to a method for obtaining a xeno-free hBS cell line, the method comprising the steps of:
i) removing the zona pellucida from a blastocyst to obtain trophectoderm-enclosed inner cell mass by a xeno-free procedure,
ii) at least partly removing the trophectoderm to obtain isolated inner cell mass cells by a xeno-free procedure,
iii) placing the inner cell mass cells on a layer of human feeder cells in a xeno-free medium,
iv) co-culturing of the inner cell mass cells with human feeder cells for a time period of from about 5 days to about 50 days in a xeno-free medium,
v) releasing the inner cell mass cells or cells derived thereof from trophectoderm overgrowth, if any, by a xeno-free procedure,
vi) selectively, transferring the inner cell mass cells or cells derived thereof to a fresh layer of human feeder cells in a xeno-free medium to obtain xeno-free hBS cells,
vii) propagating the xeno-free hBS cells by co-culturing with human feeder cells in a xeno-free medium to obtain a xeno-free hBS cell line.

In specific embodiments, the present invention provides a method for obtaining a xeno-free hBS cell line as described above, but the starting point in the method may be any of the steps mentioned above, i.e. a method of the invention may comprise step ii)-step vii), step iii)-step vii), step iv)-step vii), step v)-step vii), step vi)-step vii) or step vii) (or step viii) as mentioned below).

As used herein the term “xeno-free” is intended to mean never exposed to, directly or indirectly, material of non-human animal origin, such as cells, tissues, and/or body fluids and derivatives thereof.

In order to maintain a xeno-free hBS cell line obtained according to the invention, the method may further comprise a step
viii) propagating the xeno-free hBS cell line by co-culturing with feeder human cells in a xeno-free medium and passaging the cells at a suitable time intervals ranging from about 2 days to about 20 days, such as, e.g., from about 4 days to about 12 days.

The chosen time interval in step viii) is dependent on the cell growth.

Step viii) mentioned above is also a specific aspect of the invention.

It may be desirable to maintain the obtained xeno-free hBS cell line in a feeder free culture system and accordingly the method according to the present invention may further comprise a step
ix) transferring the xeno-free hBS cell line to a xeno-free and feeder free culture system.

Step ix) may be performed in accordance with WO2004099394, which is hereby incorporated by reference.

Step i)

The zona pellucida is a thick extracellular matrix, rich in glycoprotein, surrounding the mammalian ovum (egg). Removal of the zona pellucida from a fertilized egg in order to obtain trophectoderm-enclosed inner cell mass in step i), can be performed by using a) an acidic solution, b) recombinant enzymes such as, e.g., hyaluronidase or pronase, or c) a mechanical procedure. The process of degradation of the zona pellucida can be followed by visual inspection in a microscope.

As used herein the term “trophectoderm-enclosed inner cell mass” is intended to mean the material obtained after subjecting the fertilized egg to step I), wherein the zona pellucida is removed by either acidic hydrolysis, enzymatic digest or a mechanical procedure.

It is contemplated that the recombinant enzymes used herein have amino acid sequences which correspond to the human amino acid sequences for these enzymes, i.e. the recombinant enzymes used herein have human amino acid sequences but are produced recombinantly.

When the zona pellucida is removed by using an acidic solution, the blastocyst is subjected to the acidic solution for about 5 seconds to about 180 seconds, such as, e.g., from about 10 seconds to about 120 seconds, from about 15 seconds to about 90 seconds, from about 20 seconds to about 60 seconds, from about 30 seconds to about 50 seconds.

Importantly, pH of the acidic solution is sufficiently low as to hydrolyse the carbohydrate structure of the zona pellucida, i.e. pH of the acidic solution is from about 2 to about 3, such as, e.g., from about 2.5 to about 2.8, such as 2.5. Any suitable acids can be used. In a preferred embodiment of the present invention, the acidic solution is Acid Tyrodes solution with pH 2.5±0.3 (Sigma).

If the zona pellucida is removed by using one or more recombinant enzymes, the blastocyst is subjected to a solution of one or more recombinant enzymes. It is contemplated that the one or more recombinant enzymes have amino acid sequences which correspond to the human amino acid sequences for these enzymes.

Examples of suitable enzymes are e.g. recombinant pronase, recombinant hyaluronidase and recombinant trypsin. Additional examples of suitable cell enzymatic solutions in step (i) are recombinant or xeno-free and potentially combining proteolytic and collagenolytic enzymes, such as e.g. Accutase™ (Chemicon) and enzymatic solutions that are recombinant or xeno-free, potentially combining proteolytic, collagenolytic, and DNase activities, such as e.g. Accumax™ (Innovative Cell Technologies).

Suitable concentrations and treatment times are given in the following: Pronase: 10 U/ml, for a time period of from about 2 to about 20 minutes, such as from about 2 to about 10 min, from about 2 to about 5 min or from about 3-4 minutes (optimal).

Hyaluronidase: 70.000 U/ml, for a time period of from about 2 to about 240 minutes. Human recombinant trypsin: 5-10.000 U, for a time period of from about 0.5 to about 30 minutes

The zona pellucida can also be removed by a mechanical procedure, wherein for instance a glass capillary used under a visual inspection in the microscope.

step ii)

After removal of the zona pellucida, what is left of the blastocyst, i.e., trophectoderm-enclosed the inner cell mass, is subjected to step ii) in order to at least partly remove the trophectoderm. Alternatively, spontaneously hatched blastocysts can be subjected directly to step ii), thereby skipping step i).

Step ii) can be performed by using a) an acidic solution, b) one or more recombinant enzymes, or c) a mechanical procedure.

When step ii) is performed by using an acidic solution, the trophectoderm-enclosed inner cell mass obtained in step i), is subjected to the acidic solution for about 5 seconds to about 180 seconds, such as, e.g., from about 10 seconds to about 120 seconds, from about 15 seconds to about 90 seconds, from about 20 seconds to about 60 seconds, from about 30 seconds to about 50 seconds.

Importantly, pH of the acidic solution is sufficiently low as to hydrolyse the proteinaceous structure of the trophectoderm, i.e. pH of the acidic solution is from about 2 to about 3, such as from about 2.5 to about 2.8, such as 2.5. Any suitable acids can be used. In a preferred embodiment of the present invention, the acidic solution is Acid Tyrodes solution with pH 2.5±0.3 (Sigma).

If the trophectoderm is at least partly removed by using one or more recombinant enzymes, the trophectoderm-enclosed inner cell mass is subjected to a solution of one or more recombinant enzymes. It is contemplated that the one or more recombinant enzymes are recombinant enzymes having an amino acid sequences corresponding to the human amino acid sequences for these enzymes.

Suitable enzymes are recombinant proteolytic enzymes, such as, e.g., serine proteases including recombinant trypsin and TrypLE™ Select. If using TrypLE™ Select, step ii) is typically performed by subjecting the trophectoderm-enclosed inner cell mass obtained in step i) to an undiluted ready-to-use concentration of TrypLE™ Select for from about 0.5 to about 10 minutes, such as, e.g., from about 0.5 to about 8 minutes, from about 0.5 to about 5 minutes, from about 1 to about 3 minutes, such as for 1.5 minutes. If using recombinant trypsin, step ii) is typically performed by subjecting the trophectoderm-enclosed inner cell mass obtained in step i) to from about 5.000 U to about 10.000 U of recombinant trypsin for from about 0.5 minutes to about 30 minutes.

Additional examples of suitable cell enzymatic solutions in step (ii) are recombinant or xeno-free, potentially combining proteolytic and collagenolytic enzymes, such as e.g. Accutase™ (Chemicon) and enzymatic solutions that are recombinant or xeno-free, potentially combining proteolytic, collagenolytic, and DNase activities, such as e.g. Accumax™ (Innovative Cell Technologies).

The trophectoderm can also be at least partly removed by a mechanical procedure, which may be performed by using glass capillaries as a cutting tool. The detection of the inner cell mass cells is easily performed visually by microscopy.

Step iii)

In step iii), the resulting material after step ii) is placed on a human feeder layer typically by using a glass capillary under a microscope. Moreover, if necessary, the human feeder layer may be contained in suitable culture dishes such as, e.g., PRIMARIA® plastic dishes. If necessary the culture surface of the dishes may be coated with a suitable matrix material provided that this matrix material is xeno-free. A material suitable for use in this context includes recombinant human gelatin

Step iv)

In step iv) of the method according to the invention the inner cell mass cells are co-cultured for a time period of from about 5 days to about 50 days, such as, e.g., from about 5 days to about 30 days, from about 5 days to about 20 days or from about 5 days to about 15 days in order to expand the cell population. In one embodiment of the present invention, the inner cell mass cells are co-cultured for a time period of 10 days in step iv).

One or more medium changes may be performed during the co-cultivation of the inner cell mass cells in step iv) by changing from about 20% to about 100%, such as, e.g., from about 30% to about 80%, from about 40% to about 60% of the medium. In one embodiment of the present invention the one or more medium changes are performed during the co-cultivation of the inner cell mass cells in step iv) by changing about 50% of the medium. The one or more medium changes may be performed at time intervals of from about 2 days to about 14 days, such as, e.g., from about 4 days to about 7 days.

Since residual trophectodermal cells may have been placed on the layer of human feeder cells when placing the inner cell mass cells in step iii) visual inspection by microscopy can be performed at regular intervals in order to see whether the trophectoderm is interfering with the growth of the inner cell mass cells or inner cell mass derived cells. The suitable time point for taking the cells to step v) is when a considerable growth have been noticed over a couple of days by inspection in the microscope.

During the propagation of the inner cell mass cells performed in step iv), some of these cells might begin their transformation into blastocyst-derived stem (BS) cells. Accordingly, the cell population obtained in step iv) may comprise inner cell mass cells as well as cells derived thereof, i.e. BS cells.

Steps v) and vi)

As mentioned in the above the inner cell mass cells and cells derived thereof may be contaminated with residual trophectoderm. If that is the case, the trophectoderm tends to surround the inner cell mass cells or cells derived thereof. The inner cell mass cells or cells derived thereof can be released from such trophectoderm overgrowth by using a) a mechanical procedure or b) one or more recombinant enzymes.

A suitable mechanical procedure is to use glass capillaries as a cutting tool. Inner cell mass cells or cells derived thereof are selectively cut out upon visual inspection in a microscope and transferred to a fresh layer of human feeder cells in a xeno-free medium to obtain xeno-free hBS cells (step vi).

Alternatively, the inner cell mass cells or cells derived thereof may be released from trophectoderm overgrowth, if any, by using one or more recombinant enzymes, such as, e.g., one or more recombinant proteolytic enzymes including recombinant trypsin and TrypLE™ Select in step v). If recombinant trypsin is used for releasing the inner cell mass cells or cells derived thereof in step v), the inner cell mass cells or cells derived thereof are typically incubated with from about 5.000 U to about 10.000 U of recombinant trypsin for from about 0.5 minutes to about 30 minutes. If TrypLE™ Select is used for releasing the inner cell mass cells or cells derived thereof in step v), the inner cell mass cells or cells derived thereof are typically incubated with an undiluted ready-to-use concentration of TrypLE™ Select for from about 0.5 to about 15 minutes.

Additional examples of suitable cell enzymatic solutions in step (v) and (vi) are recombinant or xeno-free, potentially combining proteolytic and collagenolytic enzymes, such as e.g. Accutase™ (Chemicon) and enzymatic solutions that are recombinant or xeno-free potentially combining proteolytic, collagenolytic, and DNase activities, such as e.g. Accumax™ (Innovative Cell Technologies).

After having released the inner cell mass cells or cells derived thereof from trophectoderm, if any, the inner cell mass cells or cells derived thereof are selected upon visual inspection in a microscope and transferred to a fresh layer of human feeder cells in a xeno-free medium to obtain xeno-free hBS cells (step vi).

Step vii)

In step vii) the xeno-free hBS cells are propagated by co-culturing with feeder human cells to obtain a xeno-free hBS cell line. One or more passages can be performed during the propagation of the xeno-free hBS cells in step vii), wherein hBS cells are selectively passaged upon visual inspection in a microscope. These passages can be performed by using glass capillaries as a cutting tool. Alternatively, the one or more passages in step vii) can be performed by using one or more recombinant enzymes, such as, e.g., TrypLE™ Select, recombinant trypsin, and/or recombinant collagenase.

If using TrypLE™ Select, step vii) is typically performed by using an undiluted ready-to-use concentration of TrypLE™ Select for from about 0.5 minute to about 15 minutes.

If using recombinant trypsin, step vii) is typically performed by using from about 5.000 U to about 10.000 U of recombinant trypsin for from about 0.5 minutes to about 30 minutes.

If using recombinant collagenase, step vii) is typically performed by using about 200 U/ml recombinant collagenase for from about 1 minute to about 40 minutes. Additional examples of suitable cell enzymatic solutions in step (vii) are recombinant or xeno-free potentially combining proteolytic and collagenolytic enzymes, such as e.g. Accutase™ (Chmicon) and enzymatic solutions that are recombinant or xeno-free potentially combining proteolytic, collagenolytic, and DNase activities, such as e.g. Accumax™ (Innovative Cell Technologies).

The medium used in the individual passages may be the same or different.

Step viii)

Propagation of the xeno-free hBS cell line is in accordance with the propagation described in step vii).

In one embodiment of the present invention, passage of cells in step viii) may be performed by either mechanical dissection for example by using a glass capillary as cutting tool. Alternatively, the passage of cells in step viii) may be performed by using one or more recombinant enzymes, such as, e.g., TrypLE™ Select, recombinant trypsin, and/or recombinant collagenase. Concentrations and incubation times for using TrypLE™ Select, recombinant trypsin and recombinant collagenase, respectively, is as described for step vii).

Additional examples of suitable cell enzymatic solutions in step (viii) are recombinant or xeno-free, potentially combining proteolytic and collagenolytic enzymes, such as e.g. Accutase™ (Chemicon) and enzymatic solutions that are recombinant or xeno-free, potentially combining proteolytic, collagenolytic, and DNase activities, such as e.g. Accumax™ (Innovative Cell Technologies).

step ix)

In a specific embodiment of the present invention, the obtained hBS cell line is transferred to a xeno-free, feeder free culture system in step ix), said culture system comprising a suitable xeno-free support matrix such as, e.g., recombinant human gelatin, recombinant human fibronectin, human placental extracellular matrix and a suitable xeno-free medium which may be the same or different from the xeno-free medium employed upon establishment of the hBS cell line (steps iii), iv), vi), vii)) or upon maintenance on human feeder cells (step viii)). In order to maintain undifferentiated growth of the hBS cell line, the xeno-free medium can be conditioned by human feeder cells or it can be supplemented with suitable factors, such as, e.g., recombinant bFGF in high concentrations and/or activators of the WNT pathway.

Preparation of Human Feeders

The human feeder cells used in any of steps iii), vi), vii) and viii) are obtained under xeno-free conditions. Human feeder cells are derived from healthy human tissue and can be obtained by biopsy.

The human tissue from which the human feeder cells may be derived include embryonic, fetal, neonatal, juvenile or adult tissue, and it further includes tissue derived from skin, including foreskin, umbilical chord, muscle, lung, epithelium, placenta, fallopian tube, glandula, stroma or breast. The human feeder cells may be derived from cell types pertaining to the group consisting of human fibroblasts, fibrocytes, myocytes, keratinocytes, endothelial cells and epithelial cells. Examples of specific cell types that may be used for deriving human feeder cells include embryonic fibroblasts, extraembryonic endoderm cells, extraembryonic mesoderm cells, fetal fibroblasts and/or fibrocytes, fetal muscle cells, fetal skin cells, fetal lung cells, fetal endothelial cells, fetal epithelial cells, umbilical chord mesenchymal cells, placental fibroblasts and/or fibrocytes, placental endothelial cells, post-natal human foreskin fibroblasts and/or fibrocytes, post-natal muscle cells, post-natal skin cells, post-natal endothelial cells, adult skin fibroblasts and/or fibrocytes, adult muscle cells, adult fallopian tube endothelial cells, adult glandular endometrial cells, adult stromal endometrial cells, adult breast cancer parenchymal cells, adult endothelial cells, adult epithelial cells or adult keratinocytes.

Feeder cells used in the present invention may further be immortalized or genetically modified. Immortalization of feeder cells means the acquisition of the ability to grow through an theoretically indefinite number of divisions in culture. There are several methods for doing that and one approach is to transforming the cells with e.g. viruses, retro viruses and/or by the expression of telomerase reverse transciptase protein (TERT). TERT is inactive in most cells, but when hTERT is exogenously expressed the cells are able to maintain telomere lengths sufficient to avoid replicative senescence.

In addition the feeder cells may be genetically modified having specific genes integrated to the genome. These genes may code for markers of interest or for synthesis of biomolecules known to be beneficial to the hBS cells, such as growth factors, such as e.g. bFGF (basic fibroblast growth factor).

When human feeder cells are derived from hBS cells, the cells derived from hBS cells may be fibroblasts.

In one embodiment of the present invention the human feeder cells are derived from neonatal human foreskin.

In a specific embodiment of the present invention, the human feeder cells are fibroblasts, preferably derived from human neonatal foreskin fibroblasts.

Human foreskin samples can be obtained from circumcised baby boys. Samples may be aseptically selected in a sterile suitable medium, such as in sterile IMDM medium (Invitrogen) containing 2× Gentamycin (Invitrogen). Skin explants are placed in tissue culture flasks, such as, e.g., 25 cm² primaria tissue culture flasks (Becton Dickinson), containing IMDM medium (Invitrogen), 1% penicillin-streptomyocin (Gibco Invitrogen Corporation) and 10% of human serum (Tallheden et al, 2005). After approximately 10 days, a confluent monolayer is established. The cells were serially passaged using TrypLE™ Select (Invitrogen). The inventors have found that a suitable time period between each passage is from about 2 to about 20 days and normally at least 15 passages are suitable such as maximal 20 passages. After expansion they were tested for a standard panel of human pathogens, including mycoplasma, HIV of type 1 and 2, Hepatitis of type B and C, Cytomegalovirus, Herpes Simplex Virus type 1 and 2, Epstein-Barr virus and Human Pailloma virus).

Xeno-Free Medium

The medium can be any base medium suitable for propagation of human inner cell mass cells. One suitable medium is Dulbecco's Modified Eagle's Medium (DMEM) supplemented with 1-30% v/v human serum and 2-100 ng/ml recombinant bFGF. In one embodiment the base medium comprises 20% v/v human serum. In another embodiment the base medium comprises 10 ng/ml recombinant bFGF. It is contemplated that other base mediums can be used for as long as they provide the inner cell mass cells base with nutritional ingredients in a liquid form, i.e. inorganic ingredients such as trace elements and organic ingredients such as amino acids, salts, vitamins, energy providers, carbohydrates including sugars etc. Importantly, the medium is xeno-free.

The xeno-free medium used in any of the steps iii), iv), vi), vii) and/or viii) comprises a base medium suitable for propagation of human inner cell mass cells. One suitable base medium is Dulbecco's Modified Eagle's Medium (DMEM). However other base media may work as well. In addition to a xeno-free base medium, the xenofree medium may further comprise human serum, recombinant bFGF, L-glutamine or glutamax, non-essential amino acids, β-mercaptoethanol, penicillin and/or streptomycin.

The concentration of human serum in the xeno-free medium is preferably from about 1% v/v to about 30% v/v human serum, such as, e.g., from about 10% v/v to about 30% v/v human serum, from about 15% v/v to about 25% v/v human serum, and more preferably 20% v/v human serum.

The concentration of recombinant bFGF in the xeno-free medium is preferably from about 2 ng/ml to about 100 ng/ml recombinant bFGF, such as, e.g., from about 5 ng/ml to about 50 ng/ml recombinant bFGF, from about 5 ng/ml to about 25 ng/ml recombinant bFGF, from about 5 ng/ml to about 15 ng/ml recombinant bFGF, such as, e.g., 10 ng/ml recombinant bFGF.

The concentration of L-glutamine or Glutamax® in the xeno-free medium is preferably from about 0.5 mM to about 20 mM, such as, e.g., from about 0.75 mM to about 10 mM, from about 1 mM to about 5 mM, such as, e.g. 2 mM.

The concentration of non-essential amino acids in the xeno-free medium is preferably from about 0.01 mM to about 1 mM, such as, e.g., from about 0.03 mM to about 0.8 mM, from about 0.05 mM to about 0.6 mM, from about 0.07 mM to about 0.4 mM, from about 0.09 mM to about 0.2 mM, such as, e.g. 0.1 mM.

The concentration of beta-mercaptoethanol in the xeno-free medium is preferably from about 10 μM to about 200 μM, such as, e.g., from about 25 μM to about 175 μM, from about 50 μM to about 150 μM, from about 75 μM to about 125 μM, such as, e.g., 100 μm.

The concentration of penicillin in the xeno-free medium is preferably from about 5 units/ml to about 200 units/ml, such as, e.g., from about 10 units/ml to about 150 units/ml, from about 25 units/ml to about 100 units/ml, from about 25 units/ml to about 75 units/ml, such as, e.g., 50 units/ml.

The concentration of streptomycin in the xeno-free medium is preferably from about 5 μg/ml to about 200 μg/ml, such as, e.g., from about 10 μg/ml to about 150 82 g/ml, from about 25 μg/ml to about 100 μg/ml, from about 25 μg/ml to about 75 μg/ml, such as, e.g., 50 μg/ml.

Also other xeno-free media may be used for one or more individual steps of the present invention, such as in the propagation steps (vii) and (viii). Such medium may comprise salts, vitamins, an energy source (such as glucose) minerals, and amino acids. Suitable growth factors to be added to the medium could be e.g. GABA, pipecholic acid, lithium chloride, and transforming growth factor beta (TGFβ), and bFGF. Furthermore, that medium may be chemically defined. Accordingly, the xeno-free hBS cell line derived in the present invention may be sub-cultured or propagated in media other than serum-comprising medium.

Preparation of Human Serum

Superior quality human serum (Tallheden et al, 2005) was repeatedly produced in our laboratory. The blood was tested for a number of standard pathogens at the Hospital's blood centre (Hepatitis B, C, HIV, HTLV and syphilis).

Accordingly, the human serum used in any of steps iii), iv), vi), vii) and viii) is prepared by the following steps
a) collecting healthy human blood in not-heparin coated bags,
b) agitating the not-heparin coated bags for a time period of from about 0.5 hours to about 5 hours, such as, e.g., from about 0.5 hours to about 2 hours,
c) incubating the not-heparin coated bags at a temperature of at the most 5° C. for a time period of at least 10 hours
d) optionally, selection based on clotting quality such as, e.g., absence of non-clotted fibrin, opacity of the liquid phase.
e) separating the serum from the clotted material
f) sterile filtrating the serum obtained in step d)
g) pooling serum from at least 15 donors
h) freezing serum before use.

In a specific embodiment of the present invention, the method for obtaining a xeno-free hBS cell line comprises the steps of:
1) removing the zona pellucida and at least a part of the trophectoderm from a blastocyst by incubation of the blastocyst with Acid Tyrodes Solution for a time period of from about 10 sec to about 10 min, preferably from about 30 seconds to about 60 seconds, at room temperature to obtain isolated inner cell mass cells,
2) placing the inner cell mass cells on a layer of human foreskin fibroblast feeder cells in a xeno-free medium comprising DMEM, human serum, recombinant bFGF, L-glutamine or glutamax, non-essential amino acids, β-mercaptoethanol, penicillin and streptomycin.
3) co-culturing of the inner cell mass cells with human foreskin fibroblast feeder cells in a xeno-free medium for a time period of from about 5 days to about 15 days changing at least 50% of the xeno-free medium every 3-5 days.
4) releasing the inner cell mass cells or cells derived thereof from trophectoderm overgrowth, if any, by using TrypLE™ Select (Invitrogen) as enzymatic treatment
5) selectively, transferring the inner cell mass cells or cells derived thereof to fresh layers of human foreskin fibroblast feeder cells in a xeno-free medium to obtain xeno-free hBS cells,
6) propagating the xeno-free hBS cells by co-culturing with human foreskin fibroblast feeder cells in a xeno-free medium to obtain a xeno-free hBS cell line.

In specific embodiments, the present invention provides a method for obtaining a xeno-free hBS cell line as described above, but the starting point in the method may be any of the steps mentioned above, i.e. a method of the invention may comprise step 2)-step 6), step 3)-step 6), step 4)-step 6), step 5)-step 6), or step 6) (or step 7) as mentioned below).

Maintenance of the xeno-free hBS cell line obtained in step 6) may be performed by a further step
7) propagating the xeno-free hBS cell line by co-culturing with human foreskin fibroblast feeder cells in a xeno-free medium, changing at least 50% of the xeno-free medium every 3-5 days and passaging the cells at a suitable time interval such as, e.g., every 3-8 days.

Removal of the zona pellucida in step 1) can be followed by visual inspection in a microscope.

The details and particulars regarding the preferred concentrations of the individual components in the xeno-free medium discussed above apply mutatis mutandis xeno-free medium used in steps 2), 3), 5), 6) and 7).

In step 3), visual inspection may be performed at regular intervals in order to see whether trophectoderm is interfering with the growth of the inner cell mass cells or cells derived thereof.

In one embodiment of the present invention, the inner cell mass cells or cells derived thereof are selectively transferred in step 5) by using a glass capillary as cutting tool upon visual inspection in a microscope.

The passage intervals suitable for the propagation of the xeno-free hBS cell line in step 7), depends on the cell growth and are performed by manual transfer using glass capillary.

Further Aspects

In the further aspects of the present invention described in the below, the details and particulars discussed under the main aspects above shall apply mutatis mutandis.

Another aspect of the present invention relates to a method for obtaining a xeno-free hBS cell line, comprising the steps of:
i) at least partly removing the trophectoderm from a blastocyst having no zona pellucida to obtain isolated inner cell mass cells by a xeno-free procedure,
ii) placing the inner cell mass cells on a layer of human feeder cells in a xeno-free medium,
iii) co-culturing of the inner cell mass cells with human feeder cells for a time period of from about 5 days to about 50 days in a xeno-free medium,
iv) releasing the inner cell mass cells or cells derived thereof from trophectoderm overgrowth, if any, by a xeno-free procedure,
v) selectively, transferring the inner cell mass cells or cells derived thereof to a fresh layer of human feeder cells in a xeno-free medium to obtain xeno-free hBS cells,
vi) propagating the xeno-free hBS cells by co-culturing with feeder human cells in a xeno-free medium to obtain a xeno-free hBS cell line.

Still another aspect of the present invention relates to a method for obtaining a xeno-free hBS cell line, comprising the steps of:
i) placing inner cell mass cells at least partly free of the trophectoderm on a layer of human feeder cells in a xeno-free medium,
ii) co-culturing of the inner cell mass cells with human feeder cells for a time period of from about 5 days to about 50 days in a xeno-free medium,
iii) releasing the inner cell mass cells or cells derived thereof from trophectoderm overgrowth, if any, by a xeno-free procedure,
iv) selectively, transferring the inner cell mass cells or cells derived thereof to a fresh layer of human feeder cells in a xeno-free medium to obtain xeno-free hBS cells,
v) propagating the xeno-free hBS cells by co-culturing with feeder human cells in a xeno-free medium to obtain a xeno-free hBS cell line.

Yet another aspect of the present invention relates to a method for obtaining a xeno-free hBS cell line, comprising the steps of:
i) co-culturing of inner cell mass cells at least partly free of the trophectoderm with human feeder cells for a time period of from about 5 days to about 50 days in a xeno-free medium,
ii) releasing the inner cell mass cells or cells derived thereof from trophectoderm overgrowth, if any, by a xeno-free procedure,
iii) selectively, transferring the inner cell mass cells or cells derived thereof to a fresh layer of human feeder cells in a xeno-free medium to obtain xeno-free hBS cells,
iv) propagating the xeno-free hBS cells by co-culturing with feeder human cells in a xeno-free medium to obtain a xeno-free hBS cell line.

The present invention also relates to propagation and maintenance culture of a xeno-free hBS cell line independently of the method for obtaining said xeno-free hBS cell line. The human serum based medium and human feeder cells as described herein may apply mutatis mutandis to the propagation method. More details appear from the description under step vii) and step viii).

Characterization of Xeno-Free hBS Cell Line

The present invention further relates to a xeno-free hBS cell line obtained by a method according to the invention. Such xeno-free hBS cell line has never been directly or indirectly exposed to any non-human animal material, meaning that all components in all steps of the method has not been exposed to any non-human animal material either, such as, e.g., any material derived from a non-human mammal. Accordingly, the human feeder cells used according to the present invention have been derived without any exposure to any non-human animal material. Any organic material used during establishment and maintenance of the xeno-free hBS cell line is of human origin or is synthetic, semi-synthetic or recombinant material.

A xeno-free hBS cell line according to the present invention maintains self-renewal and pluripotency for a suitable period of time and, accordingly it is stable for a suitable period of time. In the present context the term “stable” is intended to denote proliferation capacity in an undifferentiated state for more than 50 weeks, such as, e.g., more than 40 weeks, more than 30 weeks, more than 20 weeks, more than 15 weeks, when grown on a layer of human feeder cells according to the invention. Thus, a xeno-free hBS cell line according to the invention is theoretically immortal.

A xeno-free hBS cell line obtained by the method according to the method of the present invention can be used for the preparation of differentiated cells. Therefore the invention also relates to such differentiated cells.

Furthermore, a xeno-free hBS cell or cell line according to the invention is capable of undergoing freezing and thawing. In a specific embodiment, the xeno-free hBS cell line obtained in the present invention can be frozen and thawed according a vitrification method previously presented by Cellartis, WO2004098285, which is hereby incorporated by reference.

To increase homogeneity of the hBS cell cultures, the cells obtained in the present invention can be subject for clonal derivation as described in WO2005059116, which is hereby incorporated by reference.

The xeno-free hBS cell line obtained by the present invention fulfils the general requirements. The cell line has one or more of the following characteristic, notably at least 4, 5, 6, 7 or 8 of the following characteristics. Thus, the cell line
i) exhibits proliferation capacity in an undifferentiated state for more than 15 weeks when grown on mitotically inactivated feeder cells, and/or
ii) exhibits normal euploid chromosomal karyotype, and/or
iii) exhibits stable chromosomal karyotype during culture and/or
iv) maintains potential to develop into derivatives of all types of germ layers both in vitro and in vivo, and/or
v) exhibits at least two of the following molecular markers OCT-4, alkaline phosphatase, the carbohydrate epitopes SSEA-3, SSEA-4, TRA 1-60, TRA 1-81, and the protein core of a keratin sulfate/chondroitin sulfate pericellular matrix proteinglycan recognized by the monoclonal antibody GCTM-2, and/or
vi) does not exhibit molecular marker SSEA-1 or other differentiation markers, and/or
vii) retains its pluripotency and forms teratomas in vivo when injected into immuno-compromised mice, and/or
viii) is capable of differentiating.

A xeno-free hBS cell line according to the invention displays at least one, such as, e.g., at least two, at least three, at least four, at least five, or at least six of the following criteria; positive reactions for Oct-4, TRA-1-60, TRA-1-81, SSEA-3 and SSEA-4 and negative reaction for SSEA-1.

In the following, methods for characterising xeno-free hBS with respect to differentiation stage, pluripotency and karyotype are described. These methods can be used to investigate whether the hBS cells obtained according to the present invention fulfil the above-mentioned criteria.

Immunohistochemistry

A xeno-free hBS cell lines according to the invention may be analysed for the immunohistochemical markers for undifferentiated cells, Oct-4, TRA-1-60, TRA-1-81, SSEA-1, SSEA-3 and SSEA-4 in order to monitor their state of differentiation.

Alkaline Phosphatase

Alkaline phospatase and telomerase activity are often regarded as markers for undifferentiated hBS cells. Activity can be measured by any commercial kit available.

Pluripotency In Vitro

The pluripotency of xeno-free hBS cell lines can be tested by letting the colonies differentiate spontaneously in the culture dishes for approximately 3-4 weeks of culture with medium changes every 2-7 days. The colonies are analysed by immunohistochemistry in order to identify cells from the three different germlayers. Suitable antibodies may be betatubulin for ectoderm, ASMA (alpha smooth muscle actin) for mesoderm and HNF3beta (hepatic nucleofactor 3 beta) for endoderm.

Pluripotency In Vivo—Teratoma

One method to analyze if a human BS cell line has remained pluripotent is to xenograft the cells to immunodeficient mice in order to obtain tumors, teratomas. Various types of tissues found in the tumor should represent all three germlayers.

Severe combined immunodeficient (SCID)-mice, a strain that lack B- and T-lymphocytes can be used for analysis of teratoma formation. Human BS cells can be surgically placed in either testis or under the kidney capsule. In testis or kidney, BS cells can be transplanted in the range of 10 000-100 000 cells. Tumors are usually palpable after approximate 1 month. The mice are then sacrificed after 1-4 months and tumors dissected and fixed for either paraffin-or freeze-sectioning. The tumor tissue is subsequently analyzed by immunohistochemical methods. Specific markers for all three germlayers can be used.

Genetic Characterization: Karyotyping and Fluorescence In Situ Hybridization (FISH) and Telomerase Activity

Chromosomes of the xeno-free hBS cell lines to be tested can be visualized using trypsin-Giemsa or DAPI staining. For fluorescence in situ hybridization (FISH) analysis, commercially available kits containing probes for chromosomes 12, 13, 17, 18, 20, 21 and the sex chromosomes (X and Y) can be used according to the manufacturer's instruction with minor modifications. Slides can further be analyzed in an inverted microscope equipped with appropriate filters and software.

Stem cells, and blastocyst-derived stem cells may further be characterized for their activity of the enzyme telomerase, which can be tested for with e.g. a kit called Telomerase PCR ELISA kit (Roche). The kit uses the internal activity of telomerase by amplification of the product by polymerase chain reaction (PCR) and detection of it with an enzyme linked immunosorbent assay (ELISA). Telomerase activity may as well be measured by QPCR.

Genetic Characterization: QPCR

The differentiation status of the cells in the present invention can furthermore be tested by QPCR for specific genes. In the following is shortly described how this can be done: Undifferentiated or differentiated hBS cell colonies may be detached from the culture plate mechanically as whole colonies and washed in PBS and stored in −80° C. RNA may further be extracted using e.g. Qiagen RNeasy Mini Kit according to the manufacturer's instructions. Reverse transcription is performed using a suitable kit therefore, such as Bio-Rad iScript First Strand Synthesis Kit (according to the manufacturer's instructions) in a Rotorgene 3000 (Corbett Research) and the QPCR is performed under suitable conditions. All genes may be quantified in the same run and—if possible—differentiation status of several samples can be compared by calculating mathematical indices for the individual samples based on the genetic markers. (More detailed protocols are described in WO2006094798.)

Testing for a Panel of Human Pathogens

The individual components used in the herein presented invention, such as the feeder cells, the serum, and the blastocyst may prior to use, as well as the xeno-free hBS cell line once established, be tested for human pathogens, such as e.g. Mycoplasma, Human Immunodeficiency Virus type 1 and 2, Hepatitis B and C, Cytomegalovirus, Herpes Simplex Virus type 1 and 2, Epstein-Barr Virus, and Human Papilloma Virus. The absence of human pathogens is of course very important for any clinical use of the xeno-free hBS cell line and cells or other biological material derived from the cell line.

Sialic Acid Neu5Gc Testing

Xeno-free hBS cells derived in accordance with the herein presented invention may further be tested for Sialic acid Neu5Gc which is a membrane bound sugar molecule. A negative result of this test could be seen as an indication that no direct or indirect exposure to non-human animal material has occurred.

Use of Xeno-Free hBS Cells or Cell Line According to the Invention

A xeno-free hBS cell line according to the present invention can be used for the preparation of differentiated derivatives thereof, such as, e.g., progenitor cells of all three germ layers and more differentiated cells displaying characteristic features for different differentiated cell types, such as, e.g., hepatocyte-like features, cardiomyocyte-like features, neuron-like features.

GMP (Good Manufacturing Procedure) Production

A xeno-free hBS cell line according to the present invention may further be used for GMP production, such as clinical GMP production of hBS cells and/or differentiated cells thereof. In addition the method for xeno-free derivation as described herein may be performed under GMP and/or cGMP conditions to provide clinically applicable cell lines and derivatives (Martin et al Nat. Med 2005).

Medical Use

A xeno-free hBS cell line according to the invention, or differentiated derivatives thereof, can be used in medicine. For example a xeno-free hBS cell line, or differentiated derivatives thereof, may be used for the manufacture of a medicinal product for the prevention and/or treatment of pathologies and/or diseases caused by tissue degeneration. Furthermore, a xeno-free hBS cell line, or differentiated derivatives thereof, may be used for the manufacture of a medicinal product for the treatment and/or prevention of metabolic pathologies and/or diseases.

The hBS cells obtained by a method according to the invention may be used for the manufacture of a medicament for transplantation of xeno-free hBS cells into a mammal for the prevention or treatment of a disease. A specific aspect is the use of xeno-free hBS cells in autologous transplantation, i.e. only human material from the specific patient in question has been used in the preparation of the xeno-freee hBS cells or cell line.

Many different kind of diseases can be envisaged, wherein xeno-free cells or cell lines according to the invention can be suitable for use including liver diseases, cardio-vascular diseases including myocardial infarction; degenenerative diseases such as, e.g., neurodegenerative diseases including Parkinson's disease and Alzheimer's disease; diabetes.

Such a medicament may comprise undifferentiated xeno-free hBS cells or differentiated xeno-free hBS cells dispersed in a pharmaceutically acceptable medium such as an aqueous medium. The medium may comprise one or more additive selected from the group consisting of pH adjusting agents, stabilizers, preservatives, osmotic pressure adjusting agent, and physiologically acceptable salts; and/or one or more agents selected from the group consisting of therapeutically active substances, prophylactically active substances, engraftment improving agents, viability improving agents, differentiation improving agent and immunosuppressive agents.

Regenerative Medicine and Cell Therapy

Owing to their pluripotency and capacity to differentiate into fully differentiated tissue cell types and/or progenitor cell types (proliferating cell type committed towards a specific tissue type) cells as derived by the xeno-free method presented herein could prove extremely useful in regenerative medicine. After differentiating the cells along a certain biological path towards e.g. a multipotent cardiac progenitor cell, treatment of cardiac related diseases can be envisioned or e.g. after differentiating the cells towards a hepatic progenitor (as presented in e.g. WO2006034873) treatment of hepatic related diseases can be envisioned or after differentiating the cells towards a neural progenitor treatment of neural diseases, such as multiple sclerosis, hypoxic injury, ischemic injury, traumatic injury, Parkinson's disease, and demyelition disorder, can be envisioned.

Additional progenitor cell types derived from a xeno-free hBS cell line obtained as described herein may be mesodermal, with the potential to give rise to other than cardiac cell types also e.g. bone and cartilage, or endodermal with the potential to give rise to also pancreatic cell types, such as beta cells.

In order to restore function in a heart which have suffered from cardiac infarction it would be necessary to replace cardiac myocytes as well as new blood vessels. When clinically compliant hBS cell lines are available, such as hBS cell lines derived according to the method presented herein, it will be possible to use these cells for the derivation of progenitor cells that later can be transplanted and evaluated for the potential use in humans. Such progenitor cells could have the potential to in situ differentiate further into the cell type of the degenerated tissue, such as the site of a cardiac infarction.

Cells differentiated from a xeno-free hBS cell line may in additon be used for treating disorders associated with, for example, necrotic, apoptotic, damaged, dysfunctional or morphologically abnormal myocardium. Such disorders include, but are not limited to, ischemic heart disease, cardiac infarction, rheumatic heart disease, endocarditis, autoimmune cardiac disease, valvular heart disease, congenital heart disorders, cardiac rhythm disorders, and cardiac insufficiency. These differentiated cells, being able to proliferate and having a potential to differentiate into cardiac cell types (including cardiomyocytes, endothelial cells and smooth muscle cells), may therefore be suitable for treating the majority of cardiac disorders and diseases by reversing, inhibiting or preventing cardiac damage caused by ischemia resulting from myocardial infarction.

In still further aspects cells differentiated from xeno-free cells as presented herein and can be used to treat cardiac disorders characterized by abnormal cardiac rhythm, such as, for example, cardiac arrhythmia.

The treatment is preferably performed by administering a therapeutically effective dose of the cells to the heart of the subject, preferably by injection into the heart. A therapeutically effective dose is an amount sufficient to generate a beneficial or desired clinical result, which dose could be administered in one or more administrations. The injection can be administered into various regions of the heart, depending on the type of cardiac tissue repair required. The administration may be performed using a catheter-based approach after opening up the chest cavity or entry through any suitable blood vessel. The effective dose of cells can be based on factors such as weight, age, physiological status, medical history, infarct size and elapsed time following onset of ischemia. The administration of the cells preferably comprises treating the subject with an immunosuppressive regimen, preferably prior to such administration, so as to inhibit such rejection.

Other Use

A xeno-free hBS cell line obtained by a method according to the present invention, or differentiated derivatives thereof, may also be used for medical research as they are very suitable for use in in vitro models for studying human diseases, such as, e.g., human degenerative diseases.

Potential applications of xeno-free hBS cells themselves and cell lines and cell populations derived therefrom are found e. g. in the drug discovery and drug development processes in the pharmaceutical industry and in toxicity testings of all kinds of chemicals. Today, large-scale and high throughput screening of drug candidates usually relies on biochemical assays that provide information on compound binding affinity and specificity, but little or no information on function. Functional screening relies upon cell-based screens and usually uses organisms of poor clinical relevance such as bacteria or yeasts that can be produced cheaply and quickly at high volume. Successive rounds of screening use model species of greater clinical relevance, but these are more costly and the screening process is time consuming. Screening tools based on human primary cells or immortalised cell types exist, but these cells are limited in supply or usefulness due to loss of vital functions as a result of in vitro culture and transformation. The access to xeno-free hBS cells and hBS cells differentiated under engineered conditions provides a new and unique capability to conduct human cell-based assays with high capacity, but without compromising clinical relevance.

Xeno-free hBS cells can be used in high throughput screenings by combining high capacity with improved clinical significance. The ability to precisely modify the genome using gene targeting in hBS cells with or without differentiation of the genetically modified cells into various cell types allows the application of this technology to the identification of novel therapeutically active substances through primary and secondary screening.

Accordingly, in other aspects the invention relates to the user of the hBS cells obtained by a method according to the invention defined for
i) the production of monoclonal antibodies,
ii) in vitro toxicity screening,
iii) in vitro screening of potential drug substances, or
iv) identification of potential drug substances.

### FIGURE LEGENDS

FIG. 1 shows a blastocyst prior to treatment with acid Tyrode's solution to remove the zona pellucida and parts of the trophectoderm in 40 times magnification.

FIG. 2 shows a blastocyst after treatment with acid Tyrode's solution to remove the zona pellucida and parts of the trophectoderm in 40 times magnification.

FIG. 3 shows hBS cell line SA611 in A) passage 7, day 4 in 10 times magnification.

FIG. 4 shows positive reactions for the undifferentiated hBS cell markers A) ALP.

FIG. 5 shows positive reaction for the undifferentiated hBS cell marker SSEA-4 and negative reaction for the differentiated hBS cell marker SSEA-1, both in passage 6 and in 20 times magnification.

FIG. 6 shows positive reactions for undifferentiated hBS cell markers Tra 1-60 and Tra 1-81, both in passage 6 and in 20 times magnification.

FIG. 7 shows positive reactions for the endodermal marker HNF-3beta and the neuroectodermal marker beta-tubulin in passage 6 after undergoing spontaneous differentiation for 21-28 days, in 20 times magnification.

FIG. 8 shows additional morphological and immuhistochemical characterization of the xeno-free hBS cell line SA611. (A) shows the blastocyst (scale-bar 25 um). (B) shows morphology of SA611 after 12 passages under xeno-free conditions (scale ba =100 um). (C—H) shows immunofluorescence of undifferentiated SA611 after 12 passages using Oct-4 (C), SSEA-1 (D), Tra1-60 (E), Tra1-81 (F), SSEA-3 (G), SSEA-4 (H). (Please not that the images in (C) and (E) are images of a double staining using different secondary antibodies.) Scale bar=50 um in (C), (E) and (G) and 100 um in (F) and (H).

FIG. 9 shows genetic characterization of the xeno-free hBS cell line SA611 in passage 9. (A) shows that the chromosomes were diploid and normal. (B) and (C) show fluorescence in situ hybridization of selected chromosomes from SA611 which demonstrates that the cells were XY and diploid for normal chromosomes 12 and 17. (C) further shows the X chromosome in blue, the Y chromosome in gold, chromosome 13 in red, chromosome 18 in aqua and chromosome 21 n green (which is nearly impossible to visualize in black and white).

FIG. 10 shows confirmation of pluripotency of the xeno-free hBSC line SA611 in vivo in (A), (C) and (E) and in vitro in (B), (D) and (F): Histological analysis of teratomas from SA611 after 11 passages under xeno-free conditions: (A) neuroectoderm (ectoderm), (C) cartilage (mesoderm), (E) secretory epithelium (endoderm). In vitro differentiated SA611 were analysed by immunofluorescence 2-4 weeks after passaging: (B) ⊕-III-tubulin positive neurons (ectoderm), (D) ASMA positive smooth muscle actin (mesoderm), and (F) HNF3β (Foxa2)-positive cells (endoderm). Scale bars 50 μm (A, B, D, E, F), scale bar 100 μm (C).

### EXAMPLES

### Example 1

### Obtaining and Culture Xeno-Free hBS Cells

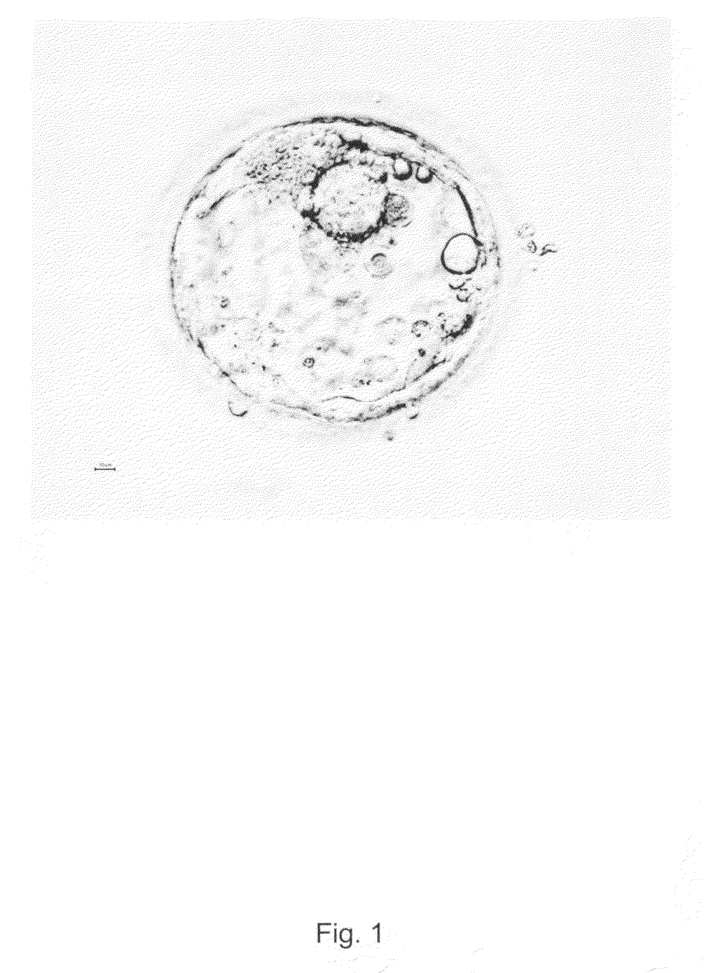

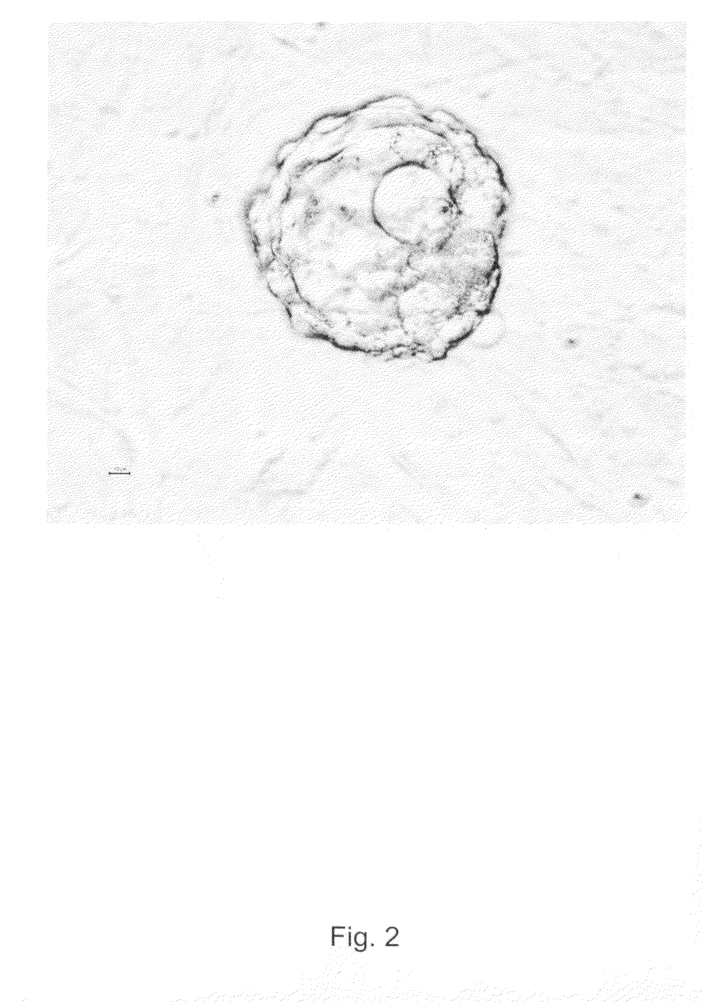

Surplus human embryo from clinical in vitro fertilization (IVF) treatment was donated after informed consent and approval from the local ethics committee at Göteborg University. The donated embryo was cultured to blastocyst until the age of 5 days in media traditionally used in IVF treatment. The blastocyst was graded according to Gardner as 4AA and according to WO2003055992 as a blastocyst of quality A (expanded blastocyst with many distinct tightly packed ICM cells and a cohesive trophectoderm with many cells). The blastocyst was treated with acid Tyrode's solution (Medicult) solution (ready-to-use concentration) for 15-30 seconds in room temperature in order to remove the zona pellucida and parts of the trophectoderm (see FIGS. 1 and 2) before placing the inner cell mass cells onto mitomycin-C inactivated xenofree human foreskin fibroblast feeders in xeno-free serum containing a DMEM with osmolarity of around 270, supplemented with 20% (v/v) human serum, 4 ng/mL human recombinant bFGF, 1% penicillin-streptomyocin, 1% Glutamax, 0.5 mmol/↑-mercaptoethanol and 1% non-essential amino acids (Gibco Invitrogen Corporation).

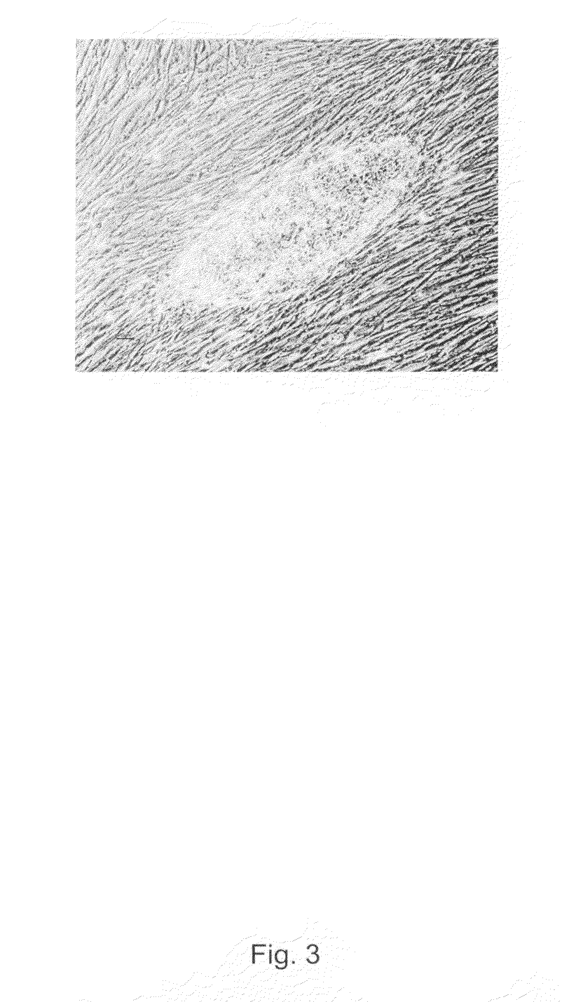

(See FIGS. 1 and 2.) The blastocyst was then incubated at 37° C. in 5% CO₂ in air. 50% of the medium was changed every 2-3 day, and after 10 days the cells were mechanically passaged to fresh hFF feeders. From passage 2 the hBS cells (cell line SA611) have been passaged mechanically using a glass capillary as cut and transfer tool. They have been passaged approximately once a week and were at the time of priority filing (October, 2005) cultured for more than 11 passages. (See FIG. 3). In total the xeno-free SA611 hBS cell line has been cultured for over 30 passages at PCT entry (October, 2006).

### Example 2

### Establishment of a Human Foreskin Fibroblast feeder Cell Line (such as Cell Line hFF003)

Human foreskin samples were aseptically collected in sterile IMDM (Invitrogen) containing 2× Gentamycin from a circumcised 8 week old boy. Skin explants were placed inside 25 cm2 primaria tissue culture flasks (Becton Dickinson) containing IMDM medium (Invitrogen), 1% penicillin-streptomyocin (Gibco Invitrogen Corporation) and 10% of human serum. After approximately 10 days, a confluent monolayer was established. The cells were serially passaged using TrypLE™ Select (Invitrogen). After expansion they were tested for a standard panel of human pathogens (mycoplasma, HIV of type 1 and 2, Hepatitis of type B and C, Cytomegalovirus, Herpes Simplex Virus type 1 and 2, Epstein-Barr virus, Human Pailloma virus).

### Example 3

### Feeder Layer Preparation

Prior to plating the xenofree human fibroblast feeders, the tissue cultured wells are coated with 0.1% recombinant human gelatin (Fibrogen) for a minimum of 1 hour at room temperature. Confluent monolayers of xenofree hFF003 (fifth to eight passage) cells grown in IMDM, 10% human serum and 1% penicillin-streptomyocin were then treated with mitomycin-C (Sigma) for 2.5 hours. Mitomycin-C treated feeders were plated on IVF wells (Becton Dickinson), 200 000 cells per 2.89 cm2 in a medium which was based on DMEM (as above) supplemented with 10% (v/v) human serum, 1% penicillin-streptomyocin, 1% Glutamax, 0.5 mmol/l β-mercaptoethanol and 1% non-essential amino acids (Gibco Invitrogen Corporation). Prior to the placing blastocysts with their inner cell mass cells and cells derived therefrom or hBS cells, the medium was changed to a DMEM (as above), now instead supplemented with 20% (v/v) human serum, 10 ng/mL human recombinant bFGF, 1% penicillin-streptomyocin, 1% Glutamax, 0.5 mmol/l β-mercaptoethanol and 1% non-essential amino acids (Gibco Invitrogen Corporation). (Same medium as described in Example 1).

### Example 4

### Preparation of Serum Based Medium

Human serum was obtained from blood-samples from at least 15 healthy individuals (Blodcentralen, Sahlgrenska University Hospital) by collecting the blood in non-heparin coated plastic bags at approximately 8° C. over night whereby the serum could be separated from the clotted material. The serum was further sterile filtered, pooled and frozen in suitable portions. (The blood prior to use was at Blodcentralen, Sahlgrenska University_Hospital tested for the standard battery of pathogens including Hepatitis B, C, HIV, HTLV and syphilis.)

The medium was further prepared by adding 20% (v/v) of the thawed serum to a DMEM (as above) together with the other ingredients as described in Example 1.

### Example 5

### Freezing and Thawing by Vitrification of Xeno-Free hBS Cells

The hBS cell line SA611 have been frozen and thawed in several passages, e.g. in passage 25 according to a method described in WO2004098285. Two solutions A and B are prepared (Solution A: Sterile filtered 10% Ethylene glycol, 10% DMSO in Cryo-PBS; Solution B: Sterile filtered 0.3M Trehalose, 20% Ethylene glycol, 10% DMSO in Cryo-PBS) Selected colonies of hBS cell line SA611 were cut in the same way as when the cells are cut for regular passage using a stem cell cutting tool (Swemed Labs International, Billdal, Sweden). The cell pieces are incubated first in 500 ml preheated (37° C.) Solution A for 1 min and then transferred to 25 ml Solution B and incubated for 30 s and then transferred again to a fresh drop of Solution B and incubated for between 20 and 30 s. The volume was about 40-50 μl. The cell pieces were aspirated into a straw prepared for vitrification and the straw was then closed with a bond. The straw was further plunged into liquid nitrogen.

Several days later straws containing frozen SA611 were thawed as described: Two solutions C and D were prepared (Solution C: Sterile filtered 0.2M Trehalose in Cryo-PBS.; Solution D: Sterile filtered 0.1M Trehalose in Cryo-PBS). Solutions C and D and hBS-medium were preheated at 37° C. The closed straws containing vitrified SA611 were removed from the liquid nitrogen tank. The straw was keep at room temperature for 10 s and then quickly thawed in a 40° C. water bath (within seconds). The straw was cut open in the plugged end using an autoclaved pair of scissors and the content pushed out from the straw into solution C using a syringe. The hBS cells were incubated for 1 min in 500 μl solution C and the transferred to 500 μl solution D and incubated for 5 min. Under a steromicroscope the hBS cell pieces were quickly rinsed in the xeno-free serum based medium and then seeded in a culture dish on top of xeno-free human fibroblast feeder cells in the serum based medium. The cells were then cultured (incubated at 37° C.) and the number of established new colonies were counted and passaged in order to verify the viability of the hBS cells after vitrification.

### Example 6

### Characterization of a Xeno-Free hBS Cell Line

Immunohistochemical and Histochemical Analysis

The hBS cell colony cultures were fixed in 4% paraformaldehyde and subsequently permabilized. After consecutive washing and blocking steps, the cells were incubated with the primary antibody overnight at 4° C. The primary antibodies used were specific for Oct-4, TRA-1-60, TRA-1-81, SSEA-1, SSEA-3 and SSEA-4 (Santa Cruz Biotechnology; Santa Cruz, Calif.; http://www.southernbiotech.com). FITC- or Cy3-conjugated secondary antibodies were used for detection. Nuclei were counterstained with DAPI (Vectashield; Vector Laboratories, Burlingame, Calif.; http://www.vectorlabs.com). The activity of alkaline phosphatese (ALP) was determined according to manufacturer's protocol (Sigma-Aldrich Stockholm, Sweden; http://www.sigmaaldrich.com).

In order to identify cells from the three different germlayers, immunohistochemical analysis was performed as outlined above with the following antibodies: For endodermal cells HNF3β (Santa Cruz Biotechnology, Santa Cruz; http://www.southernbiotech.com) was used. Mesodermal cells were detected by ASMA (company) and neuroectodermal cells was identified by β-tubulin-III mAb (Sigma-Aldrich).

Alkaline phosphatase (ALP) reactions were detected using a commercial kit and following the manufacturer's protocol (Sigma-Aldrich).

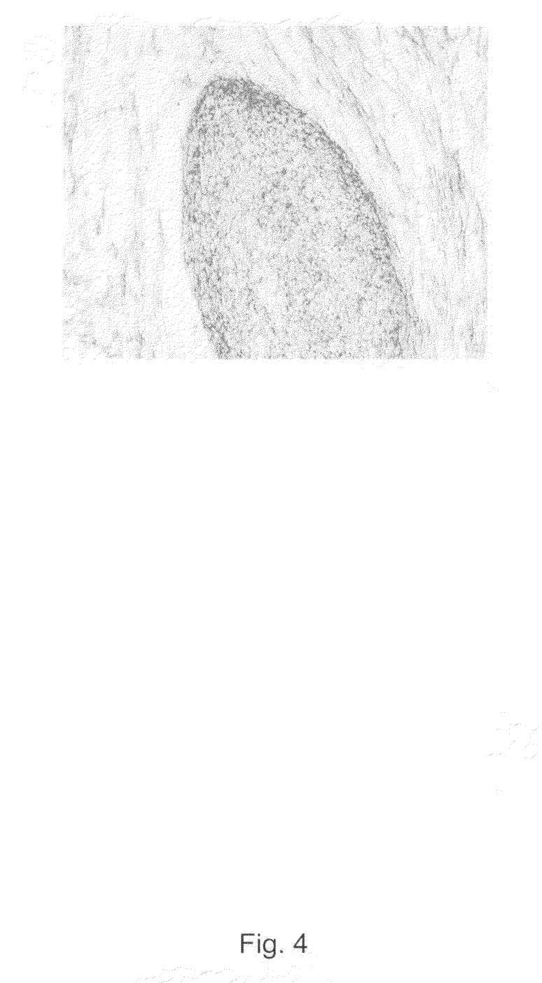

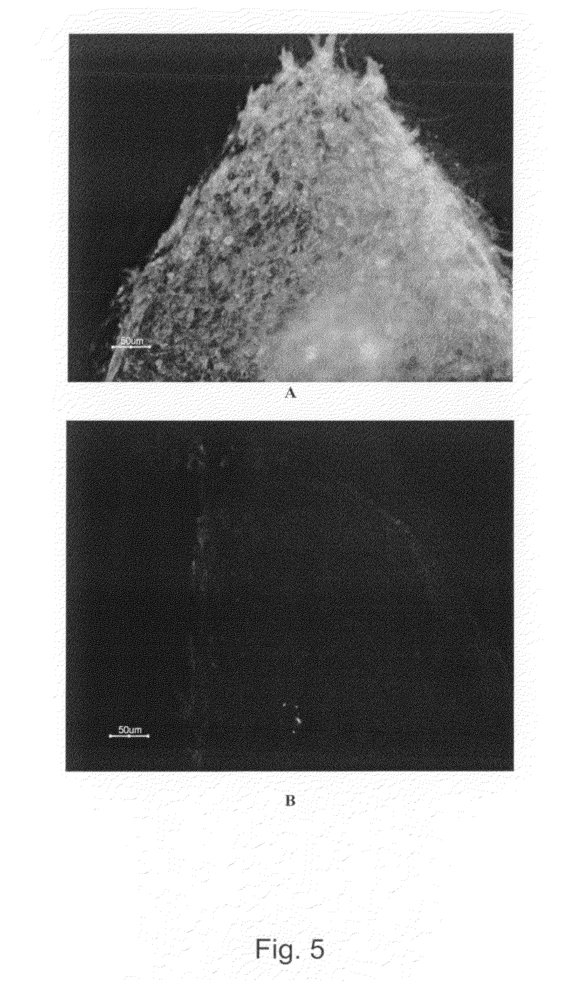

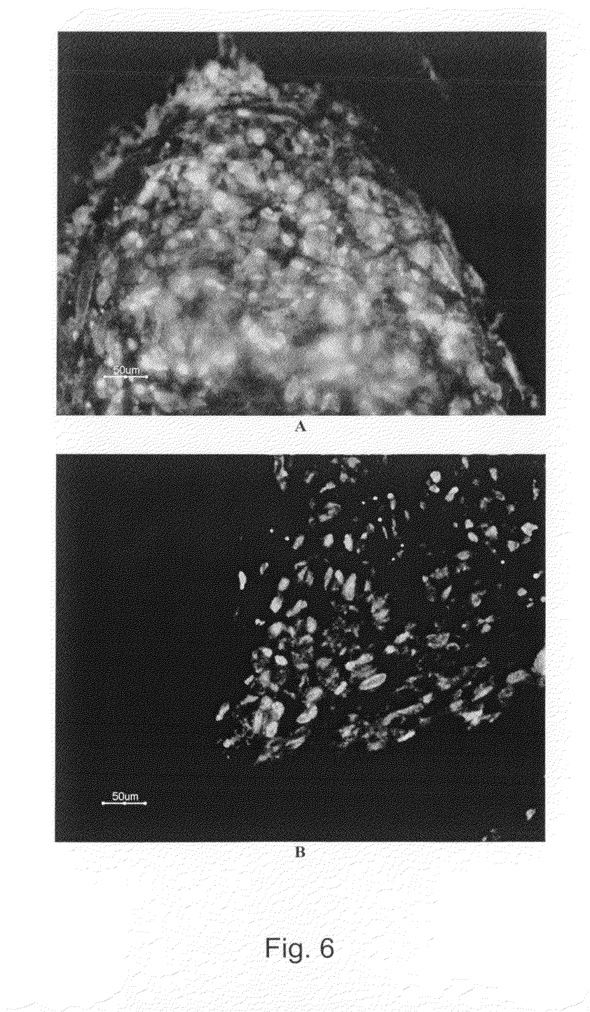

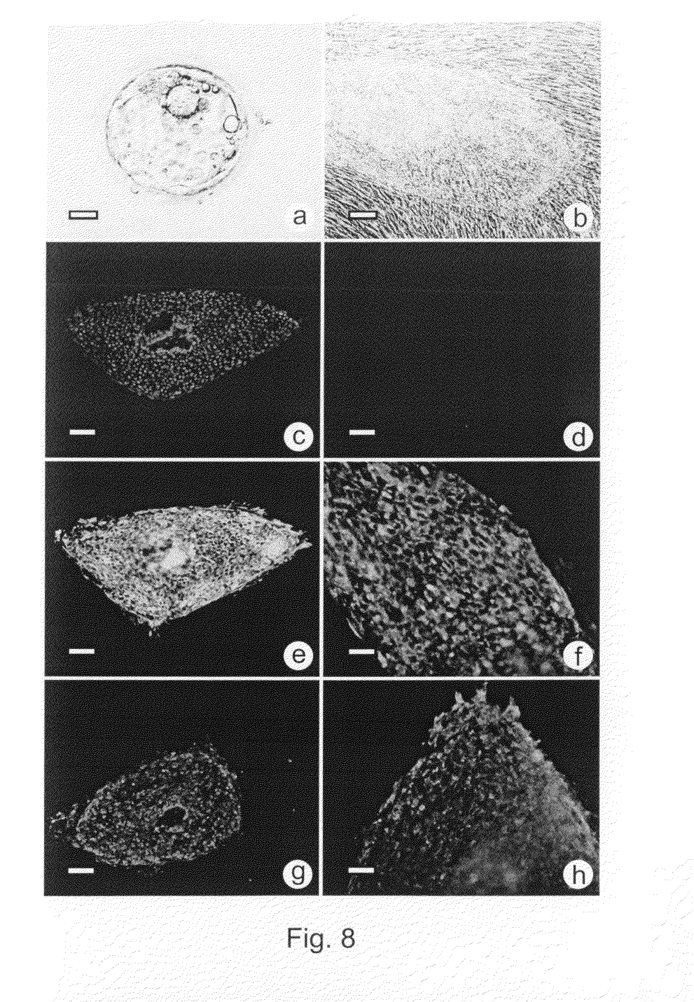

Positive reactions in undifferentiated colonies were detected for ALP (see FIG. 4), Oct-4, Tra1-60, Tra 1-81, SSEA-3, and SSEA-4 while SSEA-1 (see FIGS. 5, 6, 8) was negative. (See FIGS. 4-6.)

Karyotyping and FISH

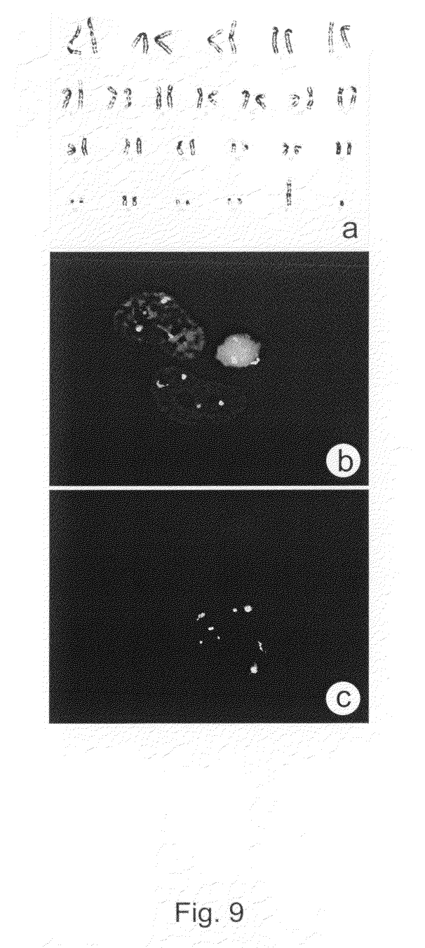

hBS cells designated for genetic characterization were retransferred to mouse embryonic fibroblasts for two passages or transferred to Matrigel plates (Becton Dickinson) and further cultured for approximately 10 days. The cells were then incubated in the presence of Calyculin A, collected by centrifugation, lysed by hypotonic treatment, and fixed using ethanol and glacial acetic acid. The chromosomes were visualized using trypsin-Giemsa or DAPI staining. For fluorescence in situ hybridization (FISH) analysis, commercially available kits containing probes for chromosomes 12, 13, 17, 18, 20, 21 and the sex chromosomes (X and Y) were used according to the manufacturer's instruction with minor modifications. The slides were analyzed in an inverted microscope equipped with appropriate filters and software (CytoVision; Applied Imaging; Santa Clara Calif.; http://www.appliedimagingcorp.com). The hBS cell line SA611 was genetically characterized at passage 9-10. The hBS cell line SA611 has a diploid normal karyotype as demonstrated by karyotype analysis. The karyotype is 46 XY. FISH analysis on selected chromosomes confirmed this finding. The karyotype of SA611 has been confirmed normal. (See FIG. 9).

Pluripotency In Vitro

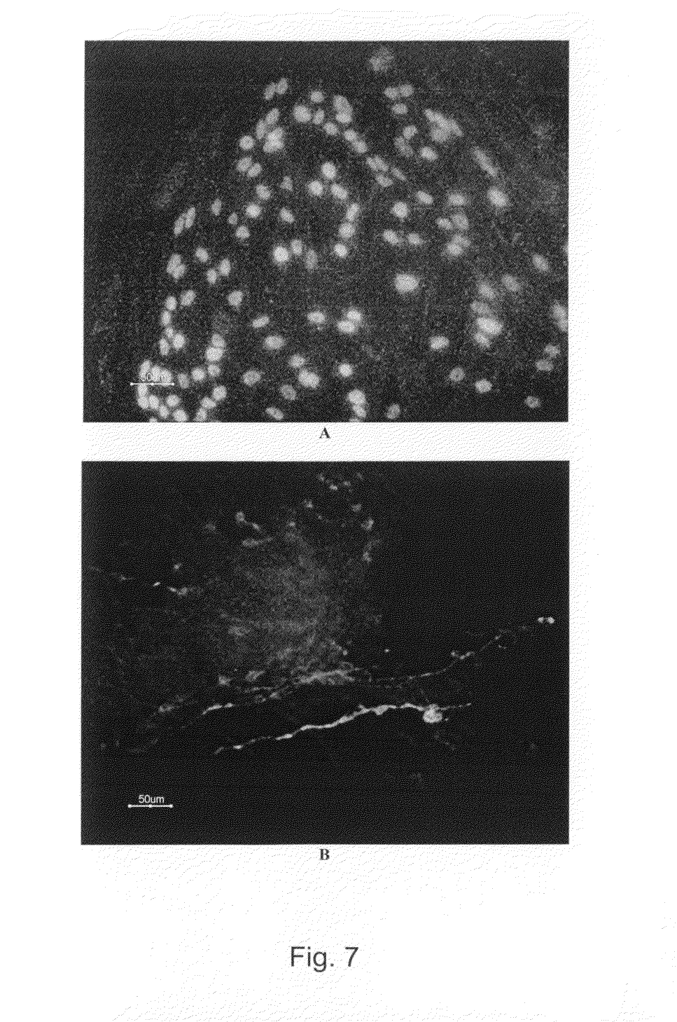

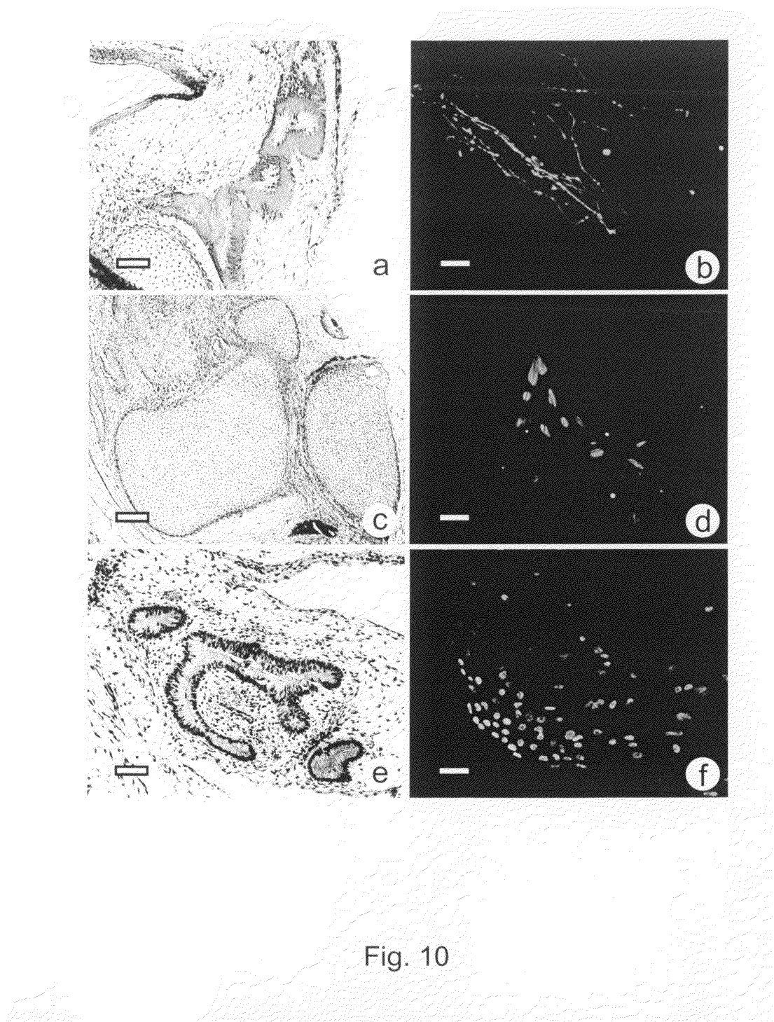

The pluripotency of SA611 was initially tested in vitro by either allowing the hBS cell colonies to spontaneously differentiate on the feeders or by transfer the undifferentiated hBS cell colonies to Matrigel™ coated plates (Becton Dickinson) on which they were allowed to spontaneously differentiate. In both cases the medium was switched to VitroHES™ (Vitrolife, Kungsbacka, Sweden) when differentiation was to be induced. After 3-4 weeks of culture, the colonies were analysed by immunohistochemistry in order to identify cells from the three different germlayers. Positive reactions were identified for the early endodermal marker HN3beta (sometimes also referred to as Foxa1), the ectodermal marker β-Tubulin and ASMA (alpha-smooth-muscle actin). (See FIG. 7 for HNF3 β and β-tubulin reactions. and FIG. 10 (B, D, F) for all three markers.)

Pluripotency In Vivo

To explore the pluripotent nature of the xeno-free hBS cell line SA611 in vivo, clusters of undifferentiated hBS cells were grafted under the kidney capsule of SCID mice. The appearance of ectodermal (neuroectoderm, FIG. 10A) mesodermal (cartilage, FIG. 10C) and endodermal (gut-like epithelium, FIG. 10E) tissues within the teratomas, demonstrated that SA611 exhibits the characteristic in vivo differentiation capacity of pluripotent hBS cells.

In summary, the xeno-free hBS cell line SA611 stably expressed the genetic and phenotypic characteristics of undifferentiated pluripotent human BS cells.

### REFERENCES

WO2004098285—Cryopreservation of human blastocyst-derived stem cells by a closed straw vitrification method

WO2003055992—A method for the establishment of a pluripotent human blastocyst-derived stem cell line

WO2005059116 Methods for clonal derivation of human blastocyst-derived stem (hBS) cell lines

WO2006094798 Use of panel of pairs of primers complementary to reporter genes of cell differentiation

WO2006034873

Improved methods for the generation of hepatocyte-like cells from human blastocyst-derived stem (hBS) cells

Martin J M, Muotri A, Gage F et al. Human embryonic stem cells express an immunogenic nonhuman sialic acid. Nat Medicin. 2005;11(2):228-232.

Xu C, Inokuma M S, Denham J et al. Feeder-free growth of undifferentiated human embryonic stem cells. Nat Biotechnol. 2001;19:971-974.

Richards M, Fong C-Y, Chan W-K et al. Human feeders support prolonged undifferentiated growth of human inner cell masses and embryonic stem cells. Nat Biotechnol. 2002;20:933-936.

Inzunza J, Gertow K, Stromberg M A et al. Derivation of human embryonic stem cell lines in serum replacement medium using postnatal human fibroblasts as feeder cells. STEM CELLS; 23:544-549.

Richards M, Tan S, Fong C Y et al. Comparative evaluation of various human feeders for prolonged undifferentiated growth of human embryonic stem cells. STEM CELLS 2003;21:546-556.

Heins N, Englund M C O, Sjöblom C et al. Derivation, characterization, and differentiation of human embryonic stem cells. STEM CELLS 2004;22:367-376.

Richards, M, Fong, C. Y, Chan, W. K, Wong, P. C, Bongso, A. Human feeders support prolonged undifferentiated growth of inner cell masses and embryonic stem cells. Nature Biotechnol. 2002; 20:933-936

Richards, M, Tan, S, Fong, C. Y, Biswas, A, Chan, W. K, Bongso, A. Comparative evaluation of various human feeders for prolonged undifferentiated growth of human embryonic stem cells, STEMCELLS 2003; 546-556

Tallheden T, van der Lee J, Brantsing C, Mansson J E, Sjogren-Jansson E, Lindahl A.

Human serum for culture of articular chondrocytes.

Cell Transplant. 2005;14(7):469-79.

Martin M J, Muotri A, Gage F, Varki A.

Human embryonic stem cells express an immunogenic nonhuman sialic acid.

Nat Med. February 2005;11(2):228-32. Epub 2005 Jan. 30.

1. A method for obtaining a xeno-free hBS cell line, the method comprising the steps of:
i) removing the zona pellucida from a blastocyst to obtain trophectoderm-enclosed inner cell mass by a xeno-free procedure,
ii) at least partly removing the trophectoderm to obtain isolated inner cell mass cells by a xeno-free procedure,
iii) placing the inner cell mass cells on a layer of human feeder cells in a xeno-free medium,
iv) co-culturing of the inner cell mass cells with human feeder cells for a time period of from about 5 days to about 50 days in a xeno-free medium,
v) releasing the inner cell mass cells or cells derived thereof from trophectoderm overgrowth, if any, by a xeno-free procedure,
vi) selectively, transferring the inner cell mass cells or cells derived thereof to a fresh layer of human feeder cells in a xeno-free medium to obtain xeno-free hBS cells, and
vii) propagating the xeno-free hBS cells by co-culturing with human feeder cells in a xeno-free medium to obtain a xeno-free hBS cell line.

2. A method according to claim 1 further comprising a step:
viii) propagating the xeno-free hBS cell line by co-culturing with feeder human cells in a xeno-free medium and passaging the cells at a suitable time intervals ranging from about 2 days to about 20 days.

3. A method according to claim 1 comprising a step:
ix) transferring the xeno-free hBS cell line to a xeno-free and feeder free culture system comprising a suitable xeno-free support matrix and a xeno-free medium.

4. A method according to claim 1, wherein step i) is performed by using a) an acidic solution, b) one or more recombinant enzymes, or c) a mechanical procedure.

5. A method according to claim 4, wherein step i) is performed by using an acidic solution.

6. A method according to claim 5, wherein step i) is performed by subjecting the blastocyst to the acidic solution for about 5 seconds to about 180 seconds.

7. A method according to claim 5, wherein the pH of the acidic solution is from about 2 to about 3.

8. A method according to claim 5, wherein the acidic solution is Acid Tyrodes solution.

9. A method according to claim 1, wherein step ii) is performed by using a) an acidic solution, b) one or more recombinant enzymes, or c) a mechanical procedure.

10. A method according to claim 9, wherein step ii) is performed by using an acidic solution.

11. A method according to claim 10, wherein step ii) is performed by subjecting the trophectoderm-enclosed inner cell mass obtained in step i) to the acidic solution for about 5 seconds to about 180 seconds.

12. A method according to or claim 10, wherein the pH of the acidic solution is from about 2 to about 3.

13. A method according to claim 10, wherein the acidic solution is Acid Tyrodes solution.

14. A method according to claim 9, wherein step ii) is performed by using one or more recombinant enzymes.

15. A method according to claim 14, wherein the one ore more recombinant enzymes are recombinant proteolytic enzymes.

16. A method according to claim 15, wherein the one or more recombinant proteolytic enzymes are selected from the group consisting of recombinant trypsin and TrypLE™ Select.

17. A method according to claim 14, wherein step ii) is performed by subjecting the trophectoderm-enclosed inner cell mass obtained in step i) to from about 5.000 U to about 10.000 U of recombinant trypsin for from about 0.5 minutes to about 30 minutes.

18. A method according to claim 14, wherein step ii) is performed by subjecting the trophectoderm-enclosed inner cell mass obtained in step i) to an undiluted ready-to-use concentration of TrypLE™ Select for from about 0.5 to about 10 minutes.

19. A method according to claim 1, wherein the time period in step iv) is from about 5 days to about 30 days in step iv).

20. A method according to claim 1, wherein one or more medium changes are performed during the co-cultivation of the inner cell mass cells in step iv) by changing from about 20% to about 100%.

21. A method according to claim 1, wherein one or more medium changes are performed during the co-cultivation of the inner cell mass cells in step iv) by changing about 50% of the medium.

22. A method according to claim 20, wherein the one or more medium changes are performed at time intervals of from about 2 days to about 14 days.

23. A method according to claim 1, wherein the inner cell mass cells or cells derived thereof are released from trophectoderm overgrowth, if any, by using a mechanical procedure in step v).

24. A method according to claim 23, wherein the mechanical procedure comprises using glass capillaries as a cutting tool.

25. A method according to claim 1, wherein the inner cell mass cells or cells derived thereof are released from trophectoderm overgrowth, if any, by using one or more recombinant enzymes in step v).

26. A method according to claim 25, wherein the one or more recombinant enzymes are recombinant proteolytic enzymes.

27. A method according to claim 26, wherein the one or more proteolytic enzymes are selected from the group consisting of recombinant trypsin and TrypLE™ Select.

28. A method according to claim 25 wherein step v) is performed by incubating the inner cell mass cells or cells derived thereof with from about 5.000 U to about 10.000 U of recombinant trypsin for from about 0.5 minutes to about 30 minutes.

29. A method according to claim 25, wherein step v) is performed by incubating the inner cell mass cells or cells derived thereof with an undiluted ready-to-use concentration of TrypLE™ Select for from about 0.5 minutes to about 15 minutes.

30. A method according to claim 1, wherein one or more passages are performed during the propagation of the xeno-free hBS cells in step vii).

31. A method according to step 30, wherein hBS cells are selectively passaged.

32. A method according to claim 30, wherein passage is performed by using a mechanical procedure.

33. A method according to claim 30, wherein passage is performed by using one or more recombinant enzymes.

34. A method according to claim 33, wherein the one or more recombinant enzymes are selected from the group consisting of TrypLE™ Select, recombinant trypsin and recombinant collagenase.

35. A method according to claim 33, wherein step vii) is performed by using from about 5.000 U to about 10.000 U of recombinant trypsin for from about 0.5 minutes to about 30 minutes.

36. A method according to claim 33, wherein step vii) is performed by using an undiluted ready-to-use concentration of TrypLE™ Select for from about 0.5 minute to about 15 minutes.

37. A method according to claim 33, wherein step vii) is performed by using about 200 U/ml recombinant collagenase for from about 1 minute to about 40 minutes.

38. A method according to claim 1, wherein the passage of cells in step viii) is performed by mechanical dissection.

39. A method according to claim 38, wherein the mechanical dissection is performed by using a glass capillary as cutting tool.

40. A method according to claim 2, wherein the passage of cells in step viii) is performed using one or more recombinant enzymes.

41. A method according to claim 40, wherein the one or more recombinant enzymes are selected from the group consisting of TrypLE™ Select, recombinant trypsin and recombinant collagenase.

42. A method according to claim 40, wherein step viii) is performed by using from about 5.000 U to about 10.000 U of recombinant trypsin for from about 0.5 minutes to about 30 minutes.

43. A method according to claim 40, wherein step viii) is performed by using an undiluted ready-to-use concentration of TrypLE™ Select for from about 0.5 minute to about 15 minutes.

44. A method according to claim 40, wherein step viii) is performed by using about 200 U/ml recombinant collagenase for from about 1 minute to about 40 minutes.

45. A method according to claim 3, wherein the xeno-free support matrix used in step ix) can be selected from the group consisting of recombinant human gelatin, recombinant human fibronectin, human placental extracellular matrix.

46. A method according to claim 3, wherein the xeno-free medium used in step ix) can be the same or different from the xeno-free medium employed in any of steps iii), iv), vi), vii) and viii).

47. A method according to claim 3, wherein the xeno-free medium used in step ix) can be conditioned by human feeder cells or it can be supplemented with suitable factors to maintain undifferentiated growth.

48. A method according to claim 47, wherein the suitable factors can be selected from the group consisting of recombinant bFGF and activators of the WNT pathway.

49. A method according to claim 2, wherein the human feeder cells used in any of steps iii), iv), vi), vii) and viii) are obtained under xeno-free conditions.

50. A method according to claim 49, wherein the human feeder cells are derived from healthy human tissue.

51. A method according to claim 49, wherein the human feeder cells are dermal fibroblasts derived from neonatal human foreskin.

52. A method according to claim 2, wherein the xeno-free medium used in any of the steps iii), iv), vi), vii) and/or viii) comprises a base medium suitable for propagation of human inner cell mass cells.

53. A method according to claim 52, wherein the base medium is DMEM.

54. A method according to claim 52, wherein the xeno-free medium further comprises from about 2 ng/ml to about 100 ng/ml recombinant bFGF.

55. A method according to claim 52, wherein the xeno-free medium further comprises 10 ng/ml recombinant bFGF.

56. A method according to claim 52, wherein the xeno-free medium further comprises from about 1% v/v to about 30% v/v human serum.

57. A method according to claim 52, wherein the xeno-free medium further comprises 20% v/v human serum.

58. A method according to claim 2, wherein the human serum used in any of steps iii), iv), vi), vii) and viii) is prepared by the following steps
a) collecting healthy human blood in not-heparin coated bags,
b) agitating the not-heparin coated bags for a time period of from about 0.5 hours to about 5 hours.
c) incubating the not-heparin coated bags at a temperature of at the most 5° C. for a time period of at least 10 hours,
d) optionally, selection based on clotting quality,
e) separating the serum from the clotted material,
f) sterile filtrating the serum obtained in step d),
g) pooling serum from at least 15 donors, and
h) freezing serum before use.

59. A method for obtaining a xeno-free hBS cell line, the method comprising the steps of:
1) removing the zona pellucida and at least a part of the trophectoderm from a blastocyst by incubation of the blastocyst with Acid Tyrodes Solution for a time period of from about 10 sec to about 10 min at room temperature to obtain isolated inner cell mass cells,
2) placing the inner cell mass cells on a layer of human foreskin fibroblast feeder cells in a xeno-free medium comprising DMEM, human serum, recombinant bFGF, L-glutamine or glutamax, non-essential amino acids, β-mercaptoethanol, penicillin and streptomycin,
3) co-culturing of the inner cell mass cells with human foreskin fibroblast feeder cells in a xeno-free medium for a time period of from about 5 days to about 15 days changing at least 50% of the xeno-free medium every 3-5 days,
4) releasing the inner cell mass cells or cells derived thereof from trophectoderm overgrowth, if any, by using TrypLE™ Select (Invitrogen) as enzymatic treatment,
5) selectively, transferring the inner cell mass cells or cells derived thereof to fresh layers of human foreskin fibroblast feeder cells in a xeno-free medium to obtain xeno-free hBS cells,
6) propagating the xeno-free hBS cells by co-culturing with human foreskin fibroblast feeder cells in a xeno-free medium to obtain a xeno-free hBS cell line.

60. A method according to claim 59 further comprising a step
7) propagating the xeno-free hBS cell line by co-culturing with human foreskin fibroblast feeder cells in a xeno-free medium, changing at least 50% of the xeno-free medium every 3-5 days and passaging the cells at a suitable time interval.

61. A xeno-free hBS cell line obtained by a method as defined in claim 1.

62. A xeno-free hBS cell line according to claim 61, which has never been directly or indirectly exposed to any non-human animal material.

63. A xeno-free hBS cell line according to 62, wherein any organic material used is of human origin or is synthetic, semi-synthetic or recombinant material.

64. A xeno-free hBS cell line according to claim 61, wherein the animal is a mammal.

65. A xeno-free hBS cell line according to claim 61 displaying at least one of the following criteria; positive reactions for Oct-4, TRA-1-60, TRA-1-81, SSEA-3 and/or SSEA-4 and negative reaction for SSEA-1.

66. A method for preparation of differentiated derivatives. of a xeno-free hBS cell line comprising utilizing the zeno-free hBS cell line of claim 61.

67. (canceled)

68. A medicinal product for the prevention and/or treatment of pathologies and/or diseases caused by-tissue degeneration comprising an effective concentration of a xeno-free hBS cell line obtained by the method of claim 1 or a differentiated derivative thereof.

69. A medicinal product for the treatment and/or prevention of metabolic pathologies and/or diseases comprising an effective concentration of a xeno-free hBS cell line obtained by the method of claim 1 or a differentiated derivative thereof.

70. (canceled)

71. A method for studying human degenerative diseases comprising utilizing a xeno-free hBS cell line obtained by the method of claim 1 or a differentiated derivative thereof.

72. A drug discovery process comprising utilizing a xeno-free hBS cell line obtained by the method of claim 1 or a differentiated derivative thereof.

73. A method for in vitro toxicity testing comprising utilizing a xeno-free hBS cell line obtained by the method of claim 1 or a differentiated derivative thereof.

74. (canceled)
